(19) United States
(12) Patent Application Publication
(10) Pub. No.: US 2017/0354375 A1
(43) Pub. Date: Dec. 14, 2017

(54) WEARABLE DEVICE AND ELECTRIC SIGNAL DETECTION UNIT THEREOF

(71) Applicant: ZENTAN TECHNOLOGY CO., LTD., New Taipei City (TW)

(72) Inventor: TONG-PIE CHEN, NEW TAIPEI CITY (TW)

(21) Appl. No.: 15/179,985

(22) Filed: Jun. 11, 2016

(51) Int. Cl. A61B 5/00; A61B 5/05

(52) U.S. Cl. CPC A61B 5/6831; A61B 5/05; A61B 2562/18; A61B 2560/0468

(57) ABSTRACT

Disclosed is a wearable device which includes a tape-like fabric body, at least one electrically conductive unit, at least one electric signal detection unit, and a data transmission unit. The electric signal detection unit and the data transmission unit are arranged on two opposite surfaces of the tape-like fabric body, and are electrically connected to each other by the electrically conductive unit. The electric signal detection unit includes a sensing portion and a connection portion, wherein the sensing portion is configured to come in contact with a body surface of a living body to detect its physiological signals, and the connection portion is configured to transmit the physiological signals to the data transmission unit. At least one of the tape-like fabric body and the sensing portion includes a heat accumulating fiber for blocking heat dissipated from the body surface of the living body to increase detection efficiency.

### BACKGROUND OF THE INVENTION

### 1. Field of the Invention

The instant disclosure relates to a physiological signal detection belt; in particular, to a wearable device for detecting physiological signals having high detection efficiency and an electric signal detection unit thereof.

### 2. Description of Related Art

The conventional detection belt for detecting physiological signals that can be worn on a living body (e.g., user's hand) by binding bands or fasteners is well known. The conventional detection belt can always detect physiological signals (e.g., heartbeat) of the living body by using its electrode assembly to contact the body surface (e.g., user's skin). The conventional detection belt not only can be used for specific patients, but also can be widely used by athletes to monitor their physiological signals.

However, there are many problems with using the conventional detection belt to detect physiological signals. One of the most serious problems is that the electrode structure of the conventional detection belt always includes dry electrodes. The dry electrodes cannot effectively detect physiological signals of the living body in a dry environment or when user's skin is too dry.

### SUMMARY OF THE INVENTION

The object of the instant disclosure is to provide a wearable device for detecting physiological signals and an electric signal detection unit thereof, which can solve the problem of the conventional detection belt which cannot effectively detect physiological signals of the living body in a dry environment or when user's skin is too dry.

In order to achieve the aforementioned objects, according to a preferred embodiment of the instant disclosure, the wearable device is configured for wearing on a living body to detect physiological signals, and comprising a tape-like fabric body, at least one electrically conductive unit, at least one electric signal detection unit, and a data transmission unit. The tape-like fabric body has a first surface and a second surface opposite to the first surface, wherein one end thereof is configured to selectively connect the other end thereof. The electrically conductive unit is arranged on the tape-like fabric body. The electric signal detection unit is arranged on the first surface of the tape-like fabric body, and includes a sensing portion and a connection portion connected to the sensing portion, wherein the sensing portion is configured to detect micro electric signals of the living body by using an external surface thereof away from the first surface to contact a body surface of the living body, and the connection portion is configured to transmit the micro electric signals to the electric signal detection unit. The data transmission unit is arranged on the second surface of the tape-like fabric body and electrically connected to the electrically conductive unit, configured to convert the micro electric signals into the digital signals. At least one of the tape-like fabric body and the sensing portion of the electric signal detection unit includes a heat accumulating fiber for blocking heat dissipated from the body surface of the living body when the sensing portion of the electric signal detection unit comes in contact with the body surface of the living body.

In order to achieve the aforementioned objects, according to a preferred embodiment of the instant disclosure, the electric signal detection unit is characterized in that it comprises an electrically conductive fabric and a heat accumulating fiber, and an external surface thereof defines a sensing portion to be in contact with a body surface of a living body to detect micro electric signals. The heat accumulating fiber is configured to block heat dissipated from the body surface of the living body when the sensing portion of the electric signal detection unit comes in contact with the body surface of the living body.

The benefits of the present invention include: at least one of the tape-like fabric body and the sensing portion of the electric signal detection unit has a heat accumulating fiber disposed therein for blocking heat dissipated from the body surface of the living body when the sensing portion of the electric signal detection unit comes in contact with the body surface of the living body, such that the detection efficiency can be increased remarkably.

### BRIEF DESCRIPTION OF THE DRAWINGS

FIG. 1 is an exploded diagram of a wearable device for detecting physiological signals according to one embodiment of the present invention;

FIG. 2 is a partially assembled diagram of the wearable device of the present invention;

FIG. 3 is an assembled diagram of the wearable device of the present invention;

FIG. 4 is a front-side schematic diagram of the wearable device of the present invention;

FIG. 5 is a cross-sectional diagram of an electric signal detection unit of the wearable device of the present invention;

FIG. 6 is an exploded diagram of the wearable device according to another embodiment of the present invention;

FIG. 7 is an exploded diagram of the wearable device according to still another embodiment of the present invention; and

FIGS. 8 to 10 are schematic diagrams illustrating locations of the heat accumulating fabric of the wearable device of the present invention.

### DETAILED DESCRIPTION OF THE PREFERRED EMBODIMENTS

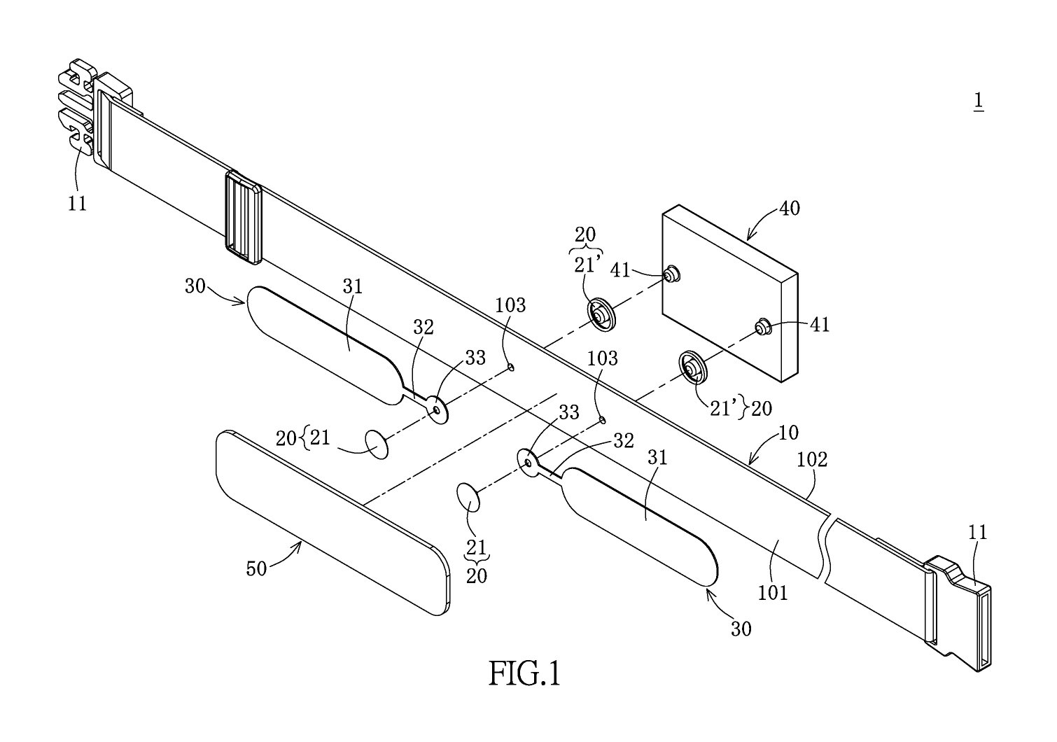

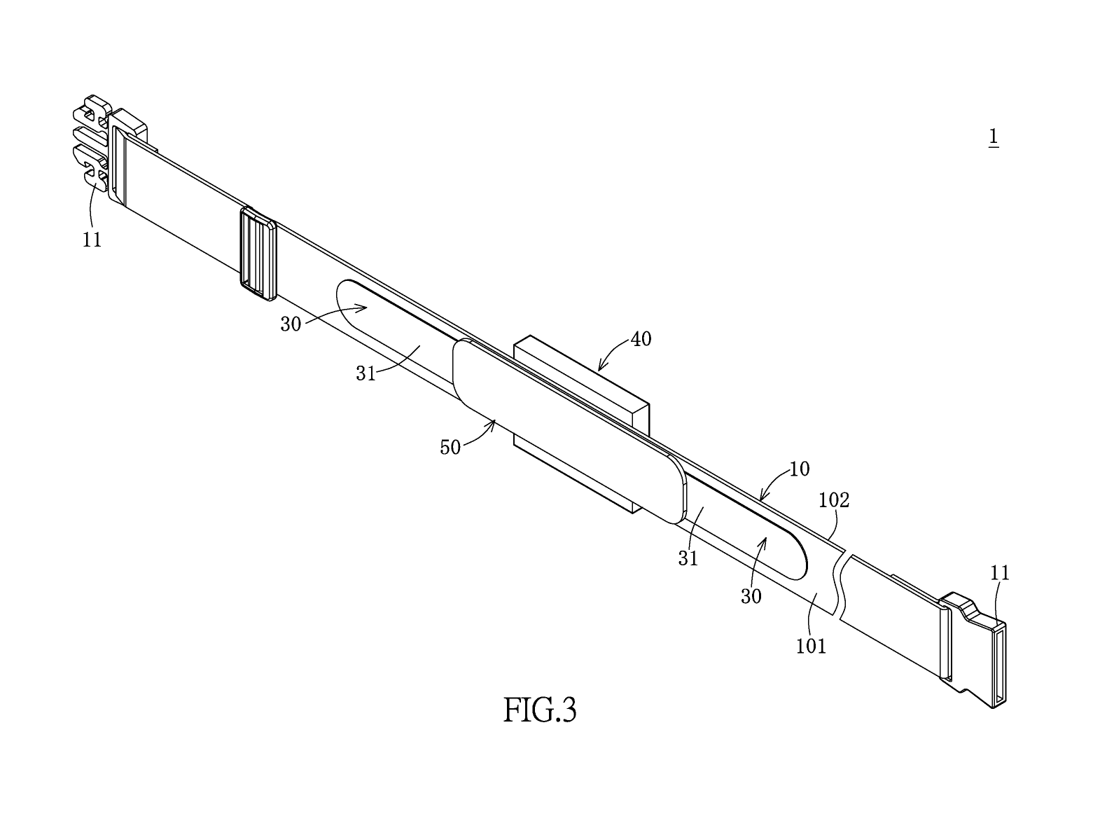

Please refer to FIGS. 1 to 5. The wearable device 1 includes a tape-like fabric body 10, two electrically conductive units 20, two electric signal detection units 30, and a data transmission unit 40. The electric signal detection units 30 and the data transmission unit 40 are arranged on two opposite surfaces of the tape-like fabric body 10 respectively. The electrically conductive units 20 are arranged through the tape-like fabric body 10 such that the electric signal detection units 30 can be electrically connected to the data transmission unit 40. The electric signal detection units 30 have an external surface away from the tape-like fabric body 10 which is configured to come in contact with a body surface of a living body to detect micro electric signals. The electric signal detection units 30 can transmit the micro electric signals to the data transmission unit 40 via the electrically conductive units 20. The data transmission unit 40 can convert the micro electric signals into digital signals and transmit the digital signals to a terminal device such as computer and smart phone via wire or wireless communications. Please note that at least one of the tape-like fabric body 10 and the electric signal detection unit 30 includes a heat accumulating fiber for blocking heat dissipated from the body surface of the living body when using the electric signal detection unit 30 to detect the micro electric signals of the living body. Thereby, sweat discharged from the body surface as a result of the heat accumulation can be used to promote the detection efficiency of the electric signal detection units 30. For the instant embodiment, specific examples of the heat accumulating fiber include, but are not limited to, polyester fiber, acrylic fiber, and acrylate fiber. In practice, the heat accumulating fiber is capable of absorbing moisture.

Specifically, the tape-like fabric body 10 has a first surface 101 and a second surface 102 opposite to the first surface 101. The two ends of the tape-like fabric body 10 each has a connection member 11 arranged thereon, and the tape-like fabric body 10 can be shaped from a linear shape into the ring-like shape by fastening the two connection members 11 together. In practice, the tape-like fabric body 10 can be worn on a living body (e.g., human body) in its ring-like shape. For the instant embodiment, each connection member 11 can be any type of conventional fastener such as, but not limited to, Velcro fastener and magnetic fastener. The tape-like fabric body 10 can be made of an elastic fiber, and selectively including a light accumulating fiber, an antibacterial fiber, a thermochromics fiber, etc., according to the end product requirements. There is no particular restriction on the material which may be used for the tape-like fabric body 10.

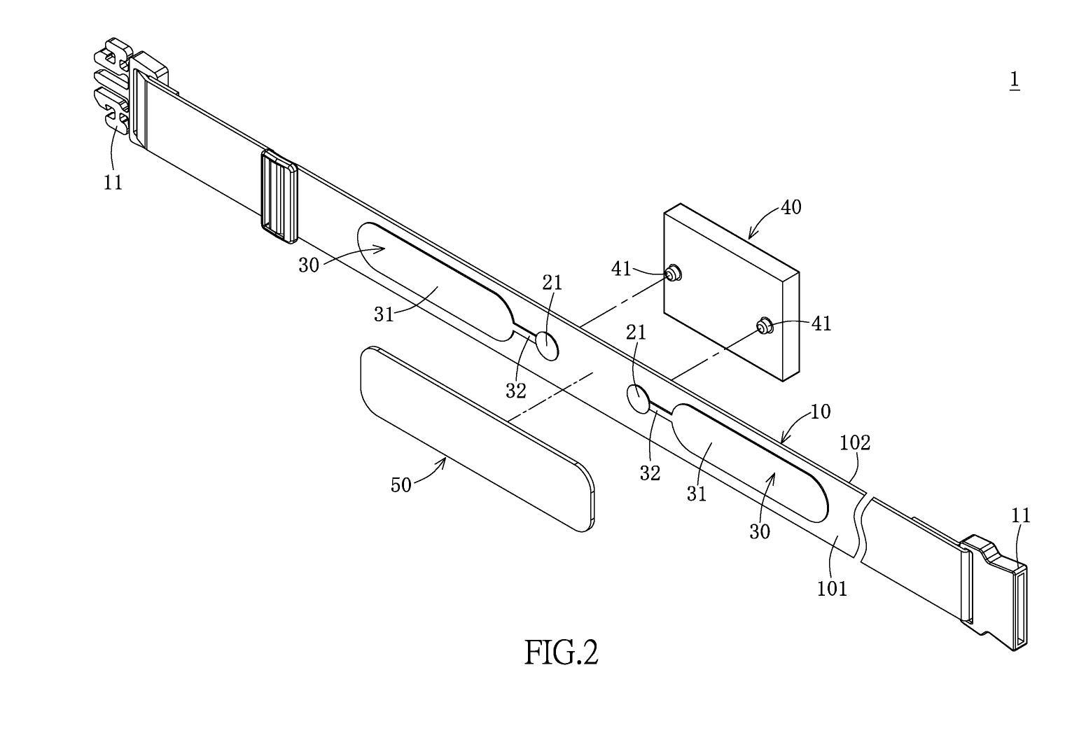

Each electrically conductive unit 20 is arranged through the tape-like fabric body 10, including a plurality of conductive members 21, 21′. The conductive members 21, 21′ are configured to be installed on the first and second surfaces 101, 102 of the tape-like fabric body 10 respectively and electrically connected to each other. The tape-like fabric body 10 is thus formed with two through holes 103. For the instant embodiment, the conductive members 21, 21′ can be metal conductive structures, wherein the design of each conductive member 21′ on the second surface 102 is in relation to a corresponding electrical connection structure 41 of the data transmission unit 40. For example, each conductive member 21′ and the corresponding electrical connection structure 41, as shown FIGS. 1 and 2, are configured to be fastened to each other.

Similarly, the design of each conductive member 21 on the first surface 101 is in relation to a corresponding connection portion 33 of the electric signal detection unit 30, as described below. Please note that, in practice, each electrically conductive unit 20 may include only one conductive member (e.g., conductive member 21′ shown in FIGS. 1 and 2) installed on the second surface 102. The conductive member 21′ is designed to pass through the tape-like fabric body 10 to be fastened to the connection portion 33.

Each of the two electric signal detection units 30 includes an electrically conductive fabric, and they are arranged on the first surface 101 of the tape-like fabric body 10 at an interval. Specifically, the two electric signal detection units 30 can be attached fixedly to the tape-like fabric body 10 by adhering, ultrasonic welding, or sewing, etc. In addition, the contour of each electric signal detection unit 30 and the fiber material disposed in each electric signal detection unit 30 can be changed according to the end product requirements. For example, each electric signal detection unit 30 can further include any functional fiber such as, but not limited to, light accumulating fiber, antibacterial fiber, and thermochromics fiber.

Each electric signal detection unit 30 has a sensing portion 31, an extension portion 32, and a connection portion 33. The extension portion 32 is connected to the sensing portion 31, and the connection portion 33 is connected to the extension portion 32. The contour of the sensing portion 31 is in relation to that of the tape-like fabric body 10, and the width of the sensing portion 31 is smaller than that of the tape-like fabric body 10. The sensing portion 31 has an external surface (unnumbered) away from the first surface 101 which is used as a sensing surface for attaching to a body surface of a living body to detect its micro electric signals. The connection portion 33 is arranged on the first surface 101 and electrically connected to the corresponding conductive member 21. Thereby, micro electric signals detected by the sensing portion 31 can be transmitted to the data transmission unit 40 by the extension portion 32 and the connection portion 33 which extends from the sensing portion 31 and the corresponding conductive members 21, 21′. The contour of the connection portion 33 of each electric signal detection unit 30 is in relation to that of the corresponding conductive member 21 on the first surface 10. For example, as shown in FIGS. 1 and 2, each connection portion 33 and the corresponding conductive member 21 on the first surface 101 are circular in shape.

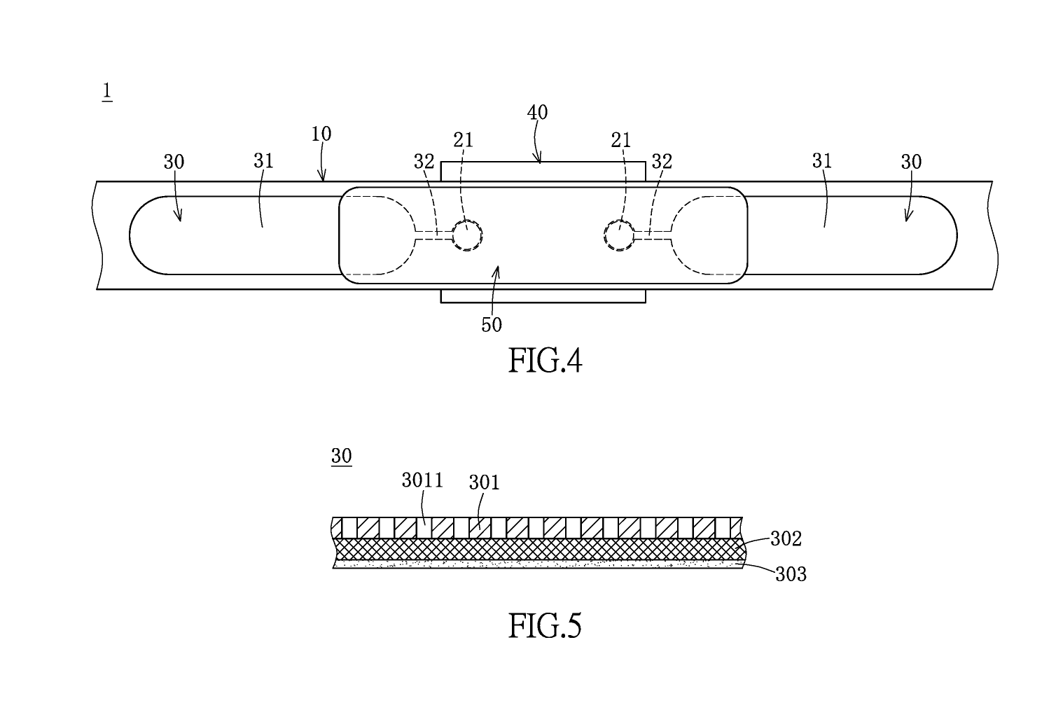

Referring to FIGS. 2 and 4, the insulating member 50 is arranged on the first surface 101 of the tape-like fabric body 10 to cover the connection portions 33. The area of the insulating member 50 is larger than the total area of the extension portions 32 and the connection portions 33, and is smaller than the area of the tape-like fabric body 10. The insulating member 50 is used to prevent the conductive members 21 on the first surface 101 from contacting with a living body. Preferably, the area of the insulating member 50 is approximately equal to the area of each electric signal detection unit 30.

Please refer to FIG. 5, which is a cross-sectional diagram of each electric signal detection unit 30. As shown in FIG. 5, each electric signal detection unit 30 is a composite material including a conductive layer 301, an interface layer 302, and a base layer 303. The conductive layer 301 and the base layer 303 are arranged on two opposite surfaces of the interface layer 302 respectively. The conductive layer 301, the interface layer 302, and the base layer 303 can be made from the same or a different fabric, and laminated together by adhering, ultrasonic welding, or sewing, etc., wherein the conductive layer 301 is used to sense and transmit the micro electric signals.

Specifically, the conductive layer 301 can be made from an electrically conductive fabric, or it can be made from an electrically conductive fabric and a heat accumulating fabric. The interface layer 302 can be made from a water-absorbing fabric, and the base layer 303 can be made from an insulating fabric. An external surface of the conductive layer 301 away from the interface layer 302 is used as the aforementioned sensing surface, and an external surface of the base layer 303 away from the interface layer 302 is connected to the first surface 101. In practice, each electric signal detection unit 30 is flexible. The conductive layer 301 can be made using any suitable method, for example, it can be made from a blended fabric including fibers and metal fines, an electrospun fabric including metal nanoparticles, a blended fabric including a conductive polymer, or a base fabric coated with a conductive material.

Preferably, the conductive layer 301, as shown in FIG. 5, can be formed with a plurality of micro-holes 3011. When the external surface of the conductive layer 301 comes in contact with a body surface of a living body, sweat discharged from the body surface will pass through the micro-holes 3011 to be absorbed by the interface layer 302 and thus to promote the detection efficiency of the conductive layer 301 upon detecting micro electric signals of the living body. In various embodiments, the base layer 303 can be made from a waterproof fabric.

In various embodiments, the conductive layer 301 with the heat accumulating fabric can selectively include a plurality of functional fibers. When the conductive layer 301 comes in contact with a body surface of a living body, the heat accumulating fabric can block heat dissipated from the body surface to assist in discharge of sweat, and sweat discharged from the body surface can be absorbed by the interface layer 302 thus to promote the detection efficiency of the conductive layer 301. In practice, the heat accumulating fiber is capable of absorbing moisture.

The data transmission unit 40 is detachably installed on the second surface 102 of the tape-like fabric body 10, and has two electrical connection structures 41 designed to be fastened to the two conductive members 21′ respectively. Thereby, the data transmission unit 40 can be electrically connected to the two conductive members 21′ on the second surface 102 by the two electrical connection structures 41, and receive the micro electric signals which are detected by the two electric signal detection units 30 from the two conductive members 21 on the first surface 101.

In other words, the micro electric signals detected by the sensing portion 31 of each electric signal detection unit 30 can be transmitted to the data transmission unit 40 by the corresponding electrically conductive unit 20 (i.e., conductive members 21, 21′ on the first and second surfaces 101, 102), and converted into output digital signals by a corresponding converting module within the data transmission unit 40. Thus, the output digital signals are then transmitted to a terminal device such as computer and smart phone. In practice, the data transmission unit 40 can selectively include functional modules based on users' requirements for converting the micro electric signals into output digital signals and then transmitting the output digital signals to a terminal device. Specific examples of the function modules include a processing module, power supply management module, signal converting module, data transmission module (e.g., bluetooth or infrared data transmission module), filtering module, and signal amplifier.

For the instant embodiment, the data transmission unit 40 is detachably installed on the second surface 102 of the tape-like fabric body 10 by fastening the two electrical connection structures 41 to the two conductive members 21′ respectively. However, in various embodiments, the data transmission unit 40 can be directly fixed on the second surface 102 of the tape-like fabric body 10, such that the wearable device 1 can be used as a one-time product. In addition, the data transmission unit 40 can be used with a replaceable battery, and it is thus reusable.

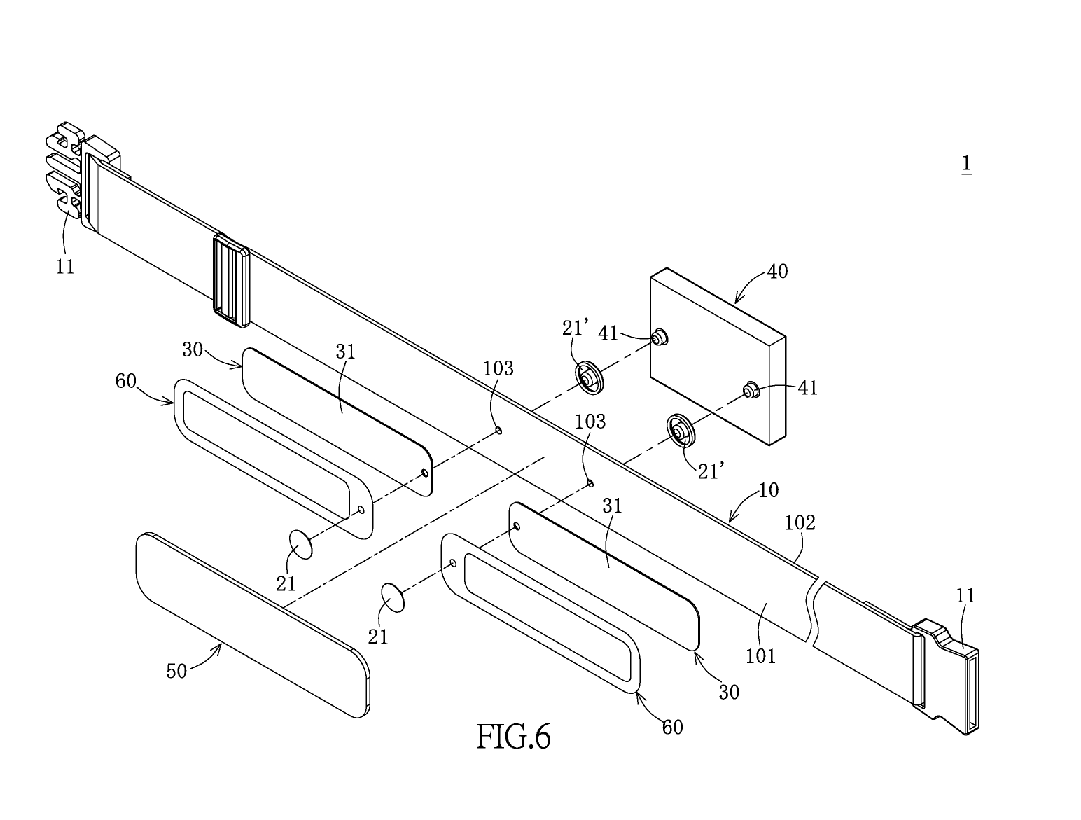

Referring to FIG. 6, the wearable device 1 further includes two anti-slip structures 60 arranged on the first surface 101 of the tape-like fabric body 10 and attached adhesively to each outer periphery of the two sensing portions 31 of the two electric signal detection units 30, wherein each anti-slip structure 60 is circular in shape. When the sensing portion 31 of each electric signal detection unit 30 comes in contact with a body surface of a living body, the frictional force between the sensing portion 31 and the body surface can be increased. Thereby, each electric signal detection unit 30 cannot break away easily from its attachment to the body surface. Specifically, each anti-slip structure 60 has a microstructure exposed from the first surface 101 of the tape-like fabric body 10.

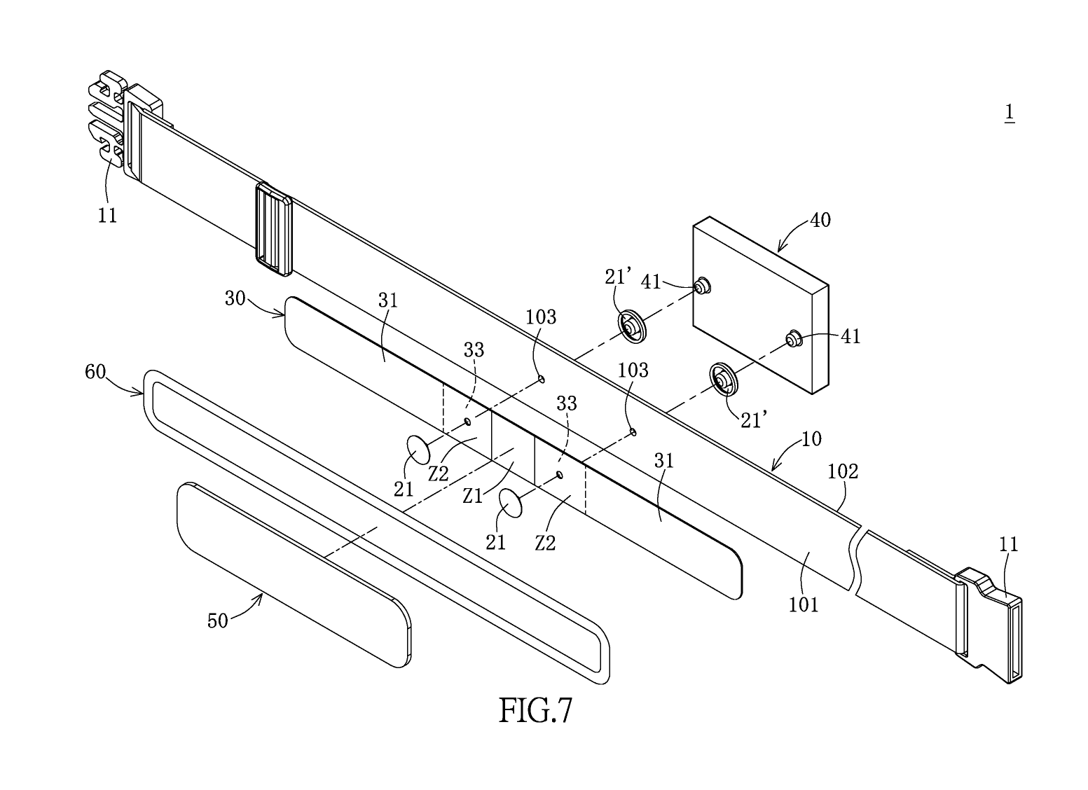

Referring to FIG. 7, the instant wearable device 1, which is different from the aforementioned wearable device 1 of FIGS. 1 thru 6 including two electric signal detection units 30, includes only one tape-like electric signal detection units 30. The electric signal detection unit 30 has a non-conductive area Z1 at its central position and two conductive areas Z2 divided by the non-conductive area Z1. Each conductive area Z2 defines a sensing portion 31 and a connection portion 33 which extends from the sensing portion 31, wherein the sensing portion 31 is configured to come into contact with a body surface of a living body, and the connection portion 33 is electrically connected to the corresponding conductive member 21 on the first surface 101.

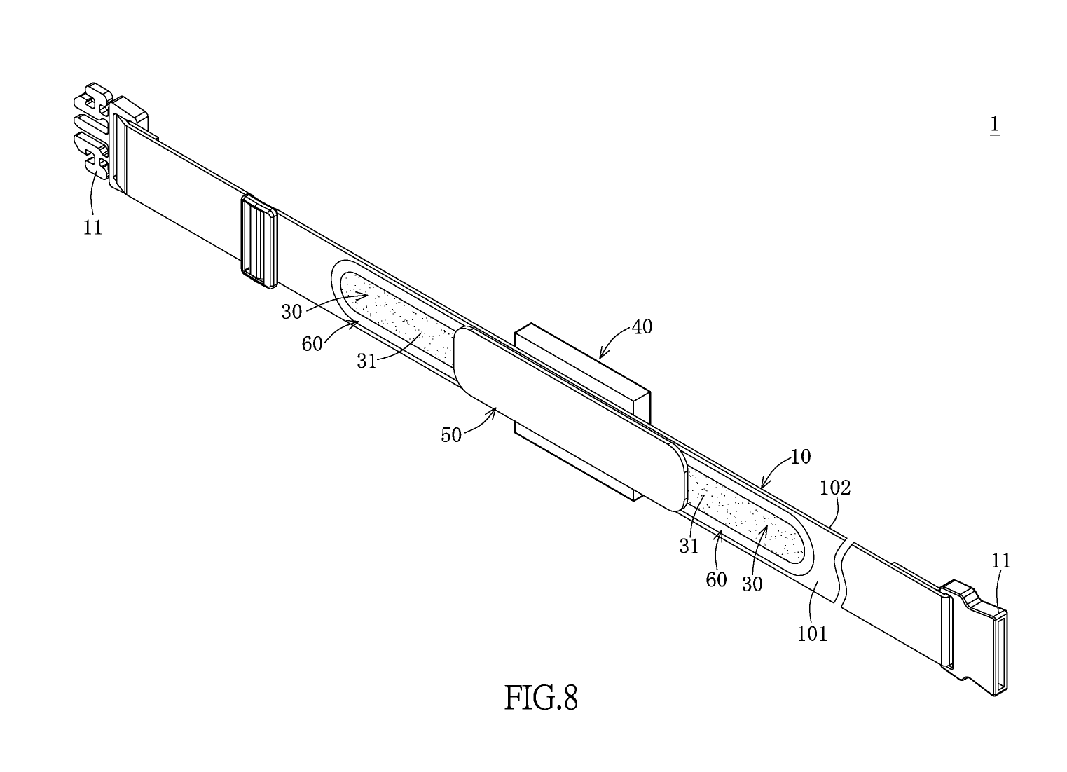

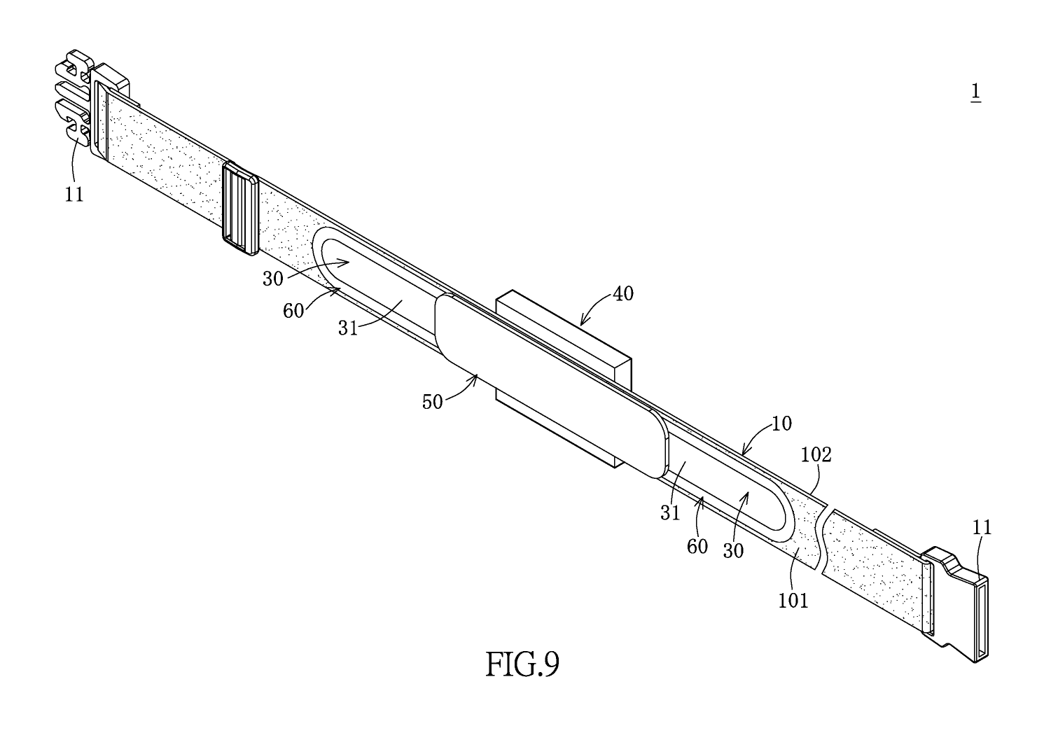

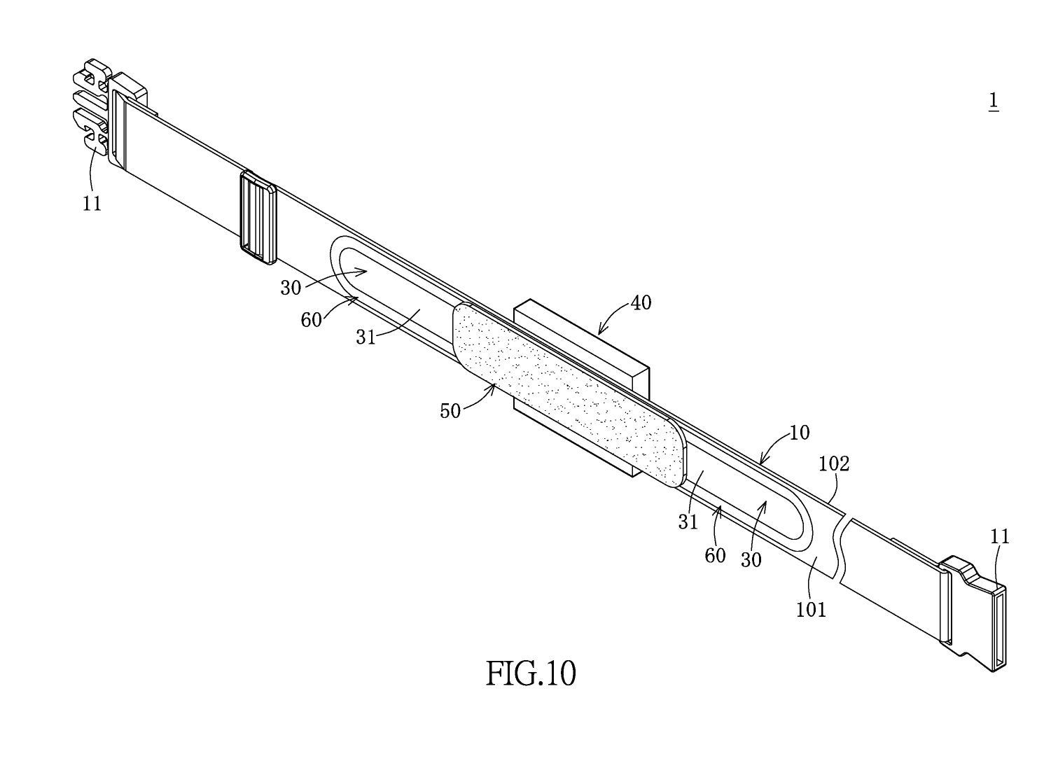

Please refer to FIGS. 8 to 10, which are schematic diagrams illustrating locations of the heat accumulating fabric of the wearable device 1. Specifically, one aspect of the heat accumulating fabric, as shown in FIG. 8, can be disposed in the sensing portion 31 of the electric signal detection units 30. Other aspects of the heat accumulating fabric, as shown in FIGS. 9 and 10, can be disposed in the tape-like fabric body 10, the insulating member 50, or the anti-slip structure 60. Thereby, when the sensing portion 31 comes in contact with a body surface of a living body, the heat accumulating fabric can cover the corresponding body surface to assist in discharge of sweat, such that sweat discharged from the body surface as a result of the heat accumulation can be used to promote the detection efficiency of the electric signal detection unit 30. Please note that any or all of the tape-like fabric body 10, the insulating member 50, or the anti-slip structure 60 can include the heat accumulating fabric.

The descriptions illustrated supra set forth simply the preferred embodiments of the instant disclosure; however, the characteristics of the instant disclosure are by no means restricted thereto. All changes, alterations, or modifications conveniently considered by those skilled in the art are deemed to be encompassed within the scope of the instant disclosure delineated by the following claims.

What is claimed is:

1. A wearable device for wearing on a living body to detect physiological signals, comprising:
a tape-like fabric body having a first surface and a second surface opposite to the first surface, wherein one end thereof is configured to selectively connect to the other end thereof;
at least one electrically conductive unit arranged to pass through the tape-like fabric body;
at least one electric signal detection unit arranged on the first surface of the tape-like fabric body, including a sensing portion and a connection portion electrically connected to the sensing portion, wherein the sensing portion is configured to detect micro electric signals of the living body by using an external surface thereof away from the first surface to come in contact with a body surface of the living body, and the connection portion is configured to transmit the micro electric signals to the electrically conductive unit; and
a data transmission unit arranged on the second surface of the tape-like fabric body and electrically connected to the electrically conductive unit, configured to convert the micro electric signals into the output digital signals;
wherein at least one of the tape-like fabric body and the sensing portion of the electric signal detection unit includes a heat accumulating fiber for blocking heat dissipated from the body surface of the living body when the sensing portion of the electric signal detection unit comes in contact with the body surface of the living body.

2. The wearable device according to claim 1, further comprising an insulating member, the electrically conductive unit includes two conductive members arranged on the first and second surfaces of the tape-like fabric body respectively, the conductive member on the second surface and a conductive structure of the data transmission unit are fasteners configured to be fastened to each other; the insulating member is arranged on the first surface of the tape-like fabric body to cover the corresponding conductive member, a connection part of the corresponding conductive member and the data transmission unit, and a part of the sensing portion of the electric signal detection unit.

3. The wearable device according to claim 2, wherein the insulating member includes a heat accumulating fiber for blocking heat dissipated from the body surface of the living body when the sensing portion of the electric signal detection unit comes in contact with the body surface of the living body.

4. The wearable device according to claim 1, wherein the electric signal detection unit is a composite material including a conductive layer, an interface layer, and a base layer, the conductive layer and the base layer are arranged on two opposite surfaces of the interface layer respectively, one side of the base layer away from the interface layer is connected to the first surface of the tape-like fabric body, and the conductive layer is formed with a plurality of micro-holes and adapted to sense and transmit the micro electric signals.

5. The wearable device according to claim 4, further comprising an anti-slip structure arranged on the first surface of the tape-like fabric body and attached adhesively to the outer periphery of the sensing portion of the electric signal detection unit, and the anti-slip structure has a microstructure exposed from the sensing portion and the first surface.

6. The wearable device according to claim 1, wherein the electric signal detection unit is a tape-like structure, having a non-conductive area and two conductive areas divided by the non-conductive area, each of the conductive areas defines a sensing portion and a connection portion which extends from the sensing portion and is electrically connected to the electrically conductive unit.

7. The wearable device according to claim 6, further comprising an insulating member arranged on the first surface of the tape-like fabric body to cover the non-conductive area and the two connection portions defined by the two conductive areas.

8. The wearable device according to claim 7, wherein the insulating member includes a heat accumulating fiber for blocking heat dissipated from the body surface of the living body when the sensing portion of the electric signal detection unit comes in contact with the body surface of the living body.

9. The wearable device according to claim 6, further comprising an anti-slip structure arranged on the first surface of the tape-like fabric body and attached adhesively to the outer periphery of the sensing portion of the electric signal detection unit, and the anti-slip structure has a microstructure exposed from the sensing portion and the first surface.

10. An electric signal detection unit characterized in that it comprises an electrically conductive fabric and a heat accumulating fiber, and an external surface thereof defines a sensing portion to be in contact with a body surface of a living body to detect micro electric signals; wherein the heat accumulating fiber is configured to block heat dissipated from the body surface of the living body when the sensing portion of the electric signal detection unit comes in contact with the body surface of the living body.

11. The wearable device according to claim 10 characterized in that it is a composite material including a conductive layer, an interface layer, and a base layer, the conductive layer and the base layer are arranged on two opposite surfaces of the interface layer respectively, one side of the base layer, away from the interface layer, is connected to the first surface of the tape-like fabric body, and the conductive layer is formed with a plurality of micro-holes and adapted to sense and transmit the micro electric signals.

12. The wearable device according to claim 11 characterized in that it is a tape-like structure and has a non-conductive area and two conductive areas divided by the non-conductive area, each of the conductive areas defines a sensing portion.
